(19) United States
(12) Patent Application Publication
(10) Pub. No.: US 2006/0134729 A1
(43) Pub. Date: Jun. 22, 2006

(54) Process for the universal detection of microorganisms and reaction environment permitting the implementation of the process

(75) Inventors: Isabelle Besson-Faure, Aubagne (FR); Jean-Pierre Hermet, Boulogne (FR); Sebastien Ribault, Lingolshein (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: HEMOSYSTEM, a corporation of France, Marseille (FR)

(21) Appl. No.: 11/136,750

(22) Filed: May 25, 2005

Related U.S. Application Data
(63) Continuation of application No. PCT/FR03/03487, filed on Nov. 25, 2003.

(30) Foreign Application Priority Data
Nov. 25, 2002 (FR) 02/14789

(51) Int. Cl. C12Q 1/04

(52) U.S. Cl. 435/34

(57) ABSTRACT

A process for detecting microorganisms present in a biological fluid including a) contacting a sample of the biological fluid with a reaction environment comprising a marking agent that is a derivative of cyanines and at least one reactant of cellular penetration of the membrane of the microorganisms, b) filtering the sample on a filter capable of retaining the marked microorganisms present in the sample, and c) detecting the marked microorganisms retained in the filter in stage (b).

### RELATED APPLICATION

This is a continuation of International Application No. PCT/FR2003/003487, with an international filing date of Nov. 25, 2003 (WO 2004/050902, published Jun. 17, 2004), which is based on French Patent Application No. 02/14789, filed Nov. 25, 2002.

### FIELD OF THE INVENTION

This invention relates to microbiology and, in particular, concerns processes for detecting and identifying microorganisms in the various environments in which they can be found.

### BACKGROUND

Numerous processes for detecting microorganisms have been developed that respond to varied requirements. Thus, the analysis of medical samples, quality control in the agrofood industry and the follow-up of water treatment can be cited.

An advantageous method of detecting microorganisms should be rapid, specific (absence of false positives), sensitive and simple to implement. It should permit the detection of living and dead microorganisms in various environments. Finally, a first identification of the types of bacteria involved would be an additional asset.

The methods of culture, on a Petri dish or in liquid phase, permit the detection of all the bacteria in a growth phase in most environments with a good sensitivity. A single bacterium suffices, in theory, to obtain a positive result after culturing and the cultures in liquid phase can be automated (G. Aubert et al., 1993). However, the time necessary to obtain the result is at times very long. Thus, the detection in blood products of strains of propionibacterium requires more than four days of culturing (M E. Brecher et al., 2001). As for mycobacterium, more than twenty days can be necessary for its detection (H. Saitoh et al., 2000). The growth of a bacterum is also heavily conditioned by the choice of the culture environment, that can be simple or enriched and that contains or does not contain inhibitors of antibacterial agents. The conditions of culturing are also specific for the strain to be detected. Thus, various incubation temperatures and aerobic or anaerobic conditions are used. The identification of microorganisms should be made with these methods in a second time after culturing. Finally, the detection of bacteria that are dead or can not be revivified is impossible with this type of technology.

The processes implementing techniques of molecular biology are rapid since several hours of incubation are sufficient to declare a positive sample and are sensitive with the possibility of detecting at least ten microorganisms per reaction.

A polymer chain reaction (PCR) permits real time detection of bacterial contamnations in a sample using fluorescent probes specific for the target DNA (Q. He et al., 2002). It is necessary to purify this sample to protect the polymerase necessary for the reaction of amplifying potential inhibitors. For example, numerous inhibitors of PCR are found in plasma (W A. Al-Soud et al., 2002). This preliminary purification stage has the result that the process of detecting microorganisms using PCR is not a process that is easy to use. Thus, in the case of a sample that contained bacteria phagocytized by leukocytes, any trace of residual DNA would bring about the positivity of the sample, which would heavily damage the specificity of the method.

The techniques of hybridization allow for universal and/or specific detection of bacteria (E B Braun-Howland et al., 1992; S. Poppert et al., 1992; S. Poppert et al., 2002). As for PCR, the preparation stage of the sample is once again a constraining and limiting stage in this method. The presence of residual nucleic acids is once again a source of false positives.

The main limitation of the techniques of molecular biology resides in the selection of the primer, whose specificity must be sufficiently great for a generic detection and nevertheless specific for the microorganisms to be detected to avoid falsely positive reactions. A mixture of different primers is generally necessary, causing technical constraints.

The immunohistochemical or immunocytochemical marking methods (enzyme-linked immunosorbent assay, ELISA) making use of an antibody directed against the bacterial wall are limited by the specificity of the antibody. In fact, at this time, no antibody permits the universal detection of microorganisms. This technique can only be used for precisely identified strains of bacteria (K. Kakinoke at al, 2001; J. Guamer et al., 2002). It also requires a particular preparaion of the cells or tissues to be analyzed comprising, e.g., stages of fixation and of cellular penetration of the sample, causing solvents of the acetone, formaldehyde and methanol types to intervene.

The microscopic methods making use of colorimetry using, e.g., GRAM colorants or vital colorants or fluorochromes allow a visual morphological identification of the type of bacterium involved in the contamination (P. Fazii et al., 2002). However, they lack sensitivity and require an elevated manipulation time as well as several days of growth of the microoranism to permit its visualization (S. Mirrett et al., 1982).

The use of cytometry permits the detection of microorganisms in a rapid and simple manner (D T. Reynolds et al., 1999; H. Okada et al., 2000). However, the limitation of this method is in the marking process. In fact, either antibodies specific for the wall of the target strain are used that do not permit the universal detection of bacteria, or DNA markers of the interalator agents type (molecules capable of inserting themselves between the plateaus formed by the base pairs of a nucleic acid). However, this latter option requires a preliminary manipuation of the bacteria to render their wall permeable to allow the marker to penetrate (D. Marie et al., 1996).

### SUMMARY OF THE INVENTION

This invention relates to a process for detecting microorganisms present in a biologcal fluid including a) contacting a sample of the biological fluid with a reaction environment including a marking agent that is a derivative of cyanines and at least one reactant of cellular penetration of the membrane of microorganisms, b) filtering the sample on a filter capable of retaining the marked microorganisms present in the sample, and c) detecting the marked microorganisms retained in the filter in stage (b).

This invention also relates to a reaction environment for marking microorganisms including a marking agent that is a derivative of cyanines and at least one cellular penetration agent of the microorganisms.

This invention further relates to a process for detecting microorganisms present in a biological fluid including a) contacting a sample of the biological fluid with a reaction environent for marking of the microorganisms including a marking agent and a reactant of cellular penetration of the membrane of the microorganisms, b) filtering the sample on a filter capable of retaining the marked microorganism present in the sample, and c) detecting the marked microoranisms retained in the filter in stage (b).

This invention still further relates to a cellular penetration reactant including Picoreen reen at 1/22000 (molecular probes); PEI at a final concentration of about 5.5 μg/ml; Diacetate chlorohexidine at a final concentration of about 4.5×10⁻⁴%; N octyl glucopyranoside at a final concentration of about 0.16%; Nisine at a final concentration of about 0.018 μg/ml; EDTA at a final concentration of about 0.45 mM; and a buffer saline phosphate (PPS) in a quantity sufficient for a selected final volume.

### BRIEF DESCRIPTION OF THE DRAWINGS

Selected aspects of the invention are illustrated with the aid of examples of implementation indicated below and accompanied by attached figures in which the concentrations are indicated as the concentrations in the penetration reactant:

FIG. 1 shows the influence of the addition of nisine on the detection of Staphylococcus epidermidis and Escherichia coli. The results are expressed as the number of bacteria detected in cytometry in solid phase (FIG. 1A) and as the percentage of bacteria detected (FIG. 1B) relative to the method of enzymatic detection.

FIG. 2 shows the effect of EDTA used solely for the detection of Staphylococcus epidermidis and Escherichia coli prepared in different test environments.

FIG. 3 illustrates the test results for the different concentrations of nisine associated with different concentrations of EDTA for improving the detection of GRAM−bacteria (Escherichia coli). The results are expressed as a percentage of detection relative to the method of enzymatic detection.

FIG. 4 illustrates the test results for different concentrations of nisine associated with a concentration of EDTA fixed at 7.5 mM for detecting the GRAM −bacteria (Escherichia coli and Serratia marcescens) and the GRAM+ bacteria (Staphylococcus epidermidis). The results are expressed as the number of bacteria detected on the filter in solid phase cytometry.

FIG. 5 illustrates the influence of the pH on the detection of E. coli with a fluorescent marker of DNA in the presence of nisine 0.2 μg/ml EDTA 7.5 mM.

FIG. 6 shows the detection of the GRAM− bacteria Escherichia coli, Serratia marcescens, Enterobacter aerogenes, Pseudomonas aeruginosa, Proteus mirabilis with a fluorescent marker of DNA in the presence of nisine 0.2 μg/ml EDTA 7.5 mM at pH 4.8.

FIG. 7 shows the results of a test of N octyl glucopyranoside as cellular penetration reactant in association with nisine 0.2 μg/ml and of EDTA 7.5 mM for improving the marking of Staphylococcus epidermidis (Gram+) and of Pseudomonas aeruginosa (Gram−). (N/E=solution of nisine 0.2 μg/ml/EDTA 7.5 mM).

FIG. 8 shows the results obtained with chlorohexidine as cellular penetration reactant for improving the marking of Escherichia coli, Pseudomonas aeruginosa and Serratia marcescens (Gram− bacterial strains) and the effect on Staphylococcus epidermidis.

FIG. 9 shows the DNA marking and the detection of bacteria (P. aeruginosa) in different environments.

FIG. 10 shows the DNA marking and detection of bacteria in chlorohexidine and demonstrates the importance of the association with NOG for increasing the permeabilizing power and the penetration of the marker. FIG. 10A shows the marking of a suspension of bacteria in PBS. FIG. 10B shows the marking of a suspension of bacteria in platelet concentrate.

FIG. 11 shows the effect of different concentrations in PEI on the detection of Serratia marcescens with a fluorescent marker of DNA.

FIG. 12 shows the effect of PEI on the DNA marking and the detection of Escherichia coli in fluorescence.

FIG. 13 shows the results of the detection of the bacteria Staphylococcus epidermidis and Escherichia coli in the presence of a marking composition comprising nisine/EDTA/CLX/NOG/PEI in different environments.

### DETAILED DESCRIPTION

We have designed a process for the universal detection of microorganisms to mitigate the disadvantages enumerated above that makes use of a marker common to all bacteria, yeasts, molds and parasites, e.g., an intercalator compound of DNA non-specific for a particular nucleic sequence.

This detection process can be applied to any biological fluid. The term “biological fluid” denotes any fluid that can contain one or several microorganisms such as, e.g., ionic environments, culture environments, physiological environments such as, e.g., blood or its derivatives such as platelet concentrates or erythrocytes or plasma and, thus, concerns various areas of application such as the analysis of medical samples, quality control in the agrofood industry or also the.follow-up of water treatment.

The process of detecting microorganisms is advantageously applied to blood or to its derivatives such as platelet concentrates or erythrocytes or plasma.

The process of marking microorganisms implements a reaction environment comprising a marking agent, cellular penetration agents that favor the molecular passage of the marking agent toward the genome of microorganisms regardless of the nature of the microorganism In a very advantageous manner the marking process allows the structure of microorganisms, especially of bacteria, to be integrally preserved.

This reaction environment allows the passage of the marking agent through:
The cytoplasmic membrane, namely, the double layer of lipid molecule and the membranous proteins of the microorganisms, whichever ones they are;
The wall of Gram positive bacteria constituted for the most part of peptidoglycane or mureine that comes into contact with the cytoplasmic membrane and that is possibly covered with a surface layer of polysaccharides; and
The external membrane of Gram negative bacteria, that contains many phospholipids, lipoproteins and lipopolysaccharides, which is separated from the cytoplasmic membrane by a periplasmic space in which the proteins are -found and that is pierced by pores. This wall is impermeable to the majority of substances with the exception of those that penetrate through the pores.

This novel process for marking microorganisms permits the universal marking of living microorganisms as well as of those that are dead or that cannot be revivified.

An analysis of the microorganisms marked in this manner can be realized, e.g., in fluorescence by microscopic methods with an epiflourescent microscope and/or cytometry in flux and/or cytometry in solid phase.

The process comprises an original preparation of microorganisms starting from samples that contain them. Various reagents are used in the same stage for penetrating the microorganisms without altering their morphology and marking them in fluorescence.

The process permits the structure of bacteria to be preserved in an integral manner for an analysis in accordance with techniques of cellular biology that may permit the visual differentiation of the large families of microorganisms: Bacilli, cocci, spores, yeasts.

This process simultaneously permits detection and morphological identification of microorganisms based on their shape and size. The process is applicable to detecting microorganisms in various physiological, culture and ionic environments.

The process advantageously and simultaneously permits detection and morphological identification of microorganisms based on the shape and size in blood or its derivatives such as platelet concentrates or erythrocytes or plasma.

The process for the universal detection of microorganisms may comprise 4 or 5 stages.

Microorganisms in suspension in water, of the buffer, of the physiological serum, of the culture environment of blood, of plasma or of blood derivatives are put in the presence of a single reaction environment comprising the intercalator agent and at least one reactant of cellular penetration. The term “reactant of cellular penetration” denotes a solution comprising at least the mixture of at least one permeabilizing agent, a detergent, an ion chelating agent and an antiseptic.

More precisely, the invention relates to a process for detecting microorganisms that may be present in a biological fluid, comprising the following stages:
a) a sample of the biological fluid is taken,
b) the sample is placed in contact with a reaction environment comprising a marking agent and a reactant of cellular penetration of the membrane of the microorganisms,
c) the sample is filtered on a filter capable of retaining the marked microorganisms possibly present in this sample, and
d) the microorganisms marked and retained in the filter in stage (c) are detected.

The marking agent is preferably an intercalator compound of DNA selected from the group comprising: cyanine compounds/derivatives, propidium iodide, orange acridine and ethidium bromide. The cyanine derivatives are selected from the group constituted of PicoGreen, SYBR green and YOPRO1. As concerns their preferred concentrations, the concentration of cyanine derivatives is between about 0.001% and about 0.5% (volume/volume), preferably between about 0.003% and about 0.05%. The concentration of propidium iodide, orange acridine or of ethidium bromide is comprised between about 0.1 μg/ml and about 100 μg/ml and preferably between about 1 μg/ml and about 40 μg/ml. The marking agent is preferably PicoGreen.

The term “preferred concentration” denotes the concentration of the product considered in the final reaction environment “biological sample and reaction environment (marking agent+ reactant of cellular penetration)”. Those skilled in the art knows how to readily adapt the concentration of the various constituents of the penetration reactant, e.g., in a concentrated mother solution.

The reactant of cellular penetration of microorganisms is preferably a solution comprising at least the mixture of at least a permeabilizing agent, a detergent, an ion chelating agent and an antiseptic.

The percentages (by weight) of the permeabilizing agent, the detergent, the ion chelating agent and the antiseptic in the final reactant are between the 1·10⁻⁴%/0.03%/0. 02%/6·10⁻⁴% and about 2.5·10⁻³%/0.8%/0.6%/0.015%.

The permeabilizing agent is selected from polyethylene glycol (PEG), digitonine, monensine, polyethylenimine (PEI), sodium hexamethaphosphate, benzalkonium chloride and the like.

The preferred concentration of these permeabilizing agents are as follows:
the concentration of PEG is between about 0.01% and about 1% and preferably between about 0.05% and about 0.5%;
the concentration of digitonine is between about 0.01 μg/ml and about 10 μg/ml and preferably between about 0.05 μg/ml and about 5 μg/ml;
the concentration of monensine is between about 0.1 μg/ml and about 5 μg/ml and preferably between about 0.5 μg/ml and about 1 μg/ml;
the concentration of PEI is between about 1 μg/ml and about 400 μg/ml and preferably between about 5 μg/ml and about 120 μg/ml;
the concentration of sodium hexametaphosphate is between about 0.005% and about 1% and preferably between about 0.01% and about 0.1%;
the concentration of benzalkonium chloride is between about 0.001% and about 0.1% and preferably between about 0.005% and about 0.05%; and
the permeabilizing agent is preferably polyethylenimine (PEI).

Among the detergents, those of the following group are preferred: N-octyl β D-glucopyranoside (NOG), saponine, Tween, Triton, Igepal and CHAPS. Their preferred concentrations are described in detail below:
the concentration of saponine or of Tween is between about 0.005% and about 10% and preferably between about 0.05% and about 0.5%;
the concentration of NOG is between about 0.01% and about 10% and preferably between about 0.1% and about 0.5%;
the concentration of Triton is between about 0.0001% and about 0.05% and preferably between about 0.0008% and about 0.002%;
the concentration of Igepal is between about 0.01% and about 20% and preferably between about 1% and about 5%; and
the detergent is preferably N-octyl β D-glucopyranoside (NOG).

As for the ion chelating agent, those of the group comprising EDTA and EGTA are preferred.

The concentration of ion chelating agent is advantageously between about 0.05% and about 0.8%.

The ion chelating agent is preferably EDTA.

The concentration of EDTA is advantageously between about 0.1 mM and about 50 mM and preferably between about 0.2 mM and about 7.5 mM.

The antiseptic agent is selected from the group comprising: Betadine, cetrimide, tea plant oil, terpinene-4-ol, chlorohexidine, polymyxine B, rifampicine and the like.

The antiseptic agent is preferably chlorohexidine.

The concentration of chlorohexidine is advantageously between about 0.0005% and about 0.05% and preferably between about 0.001% and about 0.05%;
the concentration of cetrimide is between about 0.01% and about 5% and preferably between about 0.05% and about 1%;
the concentration of betadine is between about 0.0001% and about 0.001% and preferably between about 0.0005% and about 0.005%;
the concentration of tea plant oil is between about 0.0001% and about 0.1% and preferably between about 0.0005% and about 0.05%;
the concentration of terpinen-4-ol is between about 0.05% and about 10% and preferably between about 0.5% and about 5%; and
the concentration of polymixine B and of rifampicine is between about 0.1 μg/ml and about 100 μg/ml and preferably between about 1 μg/ml and about 50 μg/ml.

The penetration reactant can also comprise an enzyme or a bacteriocine.

Lysozyme is preferably used as enzyme and nisine is preferably used as bacteriocine.

The concentration of lysozyme is advantageously between about 0.5 μg/ml and about 200 μg/ml, preferably between about 0.05 μg/ml and about 20 μg/ml, and the concentration of nisine is advantageously between about 0.005 μg/ml and about 10 μg/ml, preferably between about 0.005 μg/ml and about 0.05 μg/ml.

It is also possible to use cryoprotective agents such as DMSO or ions (NaCl, KCl, MgCl₂, sodium hypochlorite) or sucrose to effectively penetrate the bacterial wall.

The concentration of DMSO is between about 0.05% and about 20% and preferably between about 0.5% and about 5%;
the concentration of sucrose is between about 0.5% and about 70% and preferably between about 5% and about 20%;
the concentration of sodium hypochloride is between about 0.001% and about 5% and preferably between about 0.005% and about 0.5%; and
the concentration of potassium citrate is between about 0.5 mM and about 200 mM and preferably between about 5 mM and about 50 mM.

Stage b) of the process for the detection of microorganisms may be realized in two sub-stages b′) and b″).

In stage b′), the sample is placed in contact with a reaction environment comprising a marking agent and a permeabilizing polymer selected from polyethylene glycol (PEG) or polyethylenimine (PEI). Polyethylenimine (PEI) is preferably used.

In stage b″), a mixture is added to the reaction environment which mixture comprises at least one detergent, an ion chelating agent, an antiseptic and another permeabilizing agent selected from nisine, digitonine, sodium hexamethaphosphate, benzalkonium chloride and the like.

When step b) of the process is realized in two stages b′) and b″), the enzyme is added to stage b″).

The invention. also relates to a reaction environment for marking microorganisms comprising a marking agent and a reactant for the cellular penetration of these microorganisms.

A preferred reactant for cellular penetration comprises:
PicoGreen at 1/22000 (molecular probes);
PEI at a final concentration of 5.5 μg/ml;
Diacetate chlorohexidine at a final concentration of 4.5×10⁻⁴%;
N octyl glucopyranoside at a final concentration of 0.16%;
Nisine at a final concentration of 0.018 μg/ml;
EDTA at a final concentration of 0.45 mM; and
the buffer saline phosphate (PPS) in a quantity sufficient for the final volume desired.

The process for the detection of microorganisms in a sample can be carried out by implementing a treatment of the sample in two stages, a first stage of marking/cellular penetration by adding to the sample a composition comprising the marking agent and a first cellular penetration reactant followed after an incubation time by a second stage in which a composition is added comprising other cellular penetration reactants.

Such a process can be implemented, e.g., in accordance with the protocol described below:

Three milliliters of the sample to be treated are incubated for 40 minutes in one milliliter of a first solution of cellular penetration/marking (PicoGreen 0.5 mm/l, PEI 60 mg/l, PBS solution). This stage is carried out at an ambient temperature under agitation.

In the second stage, seven milliliters of a composition in solution are added that permits the marking to be followed (nisine 0.2 mg/l, NOG 2.5 g/l, EDTA 1.86 g/l, chlorohexidine Diacetate 50 mg/l). Incubation is performed at ambient temperature for 20 minutes. The sample is then filtered on a char filter, e.g., of polycarbonate or polyester and analyzed with a cytometer in solid phase.

The process of detecting microorganisms in the sample can also be carried out by implementing a treatment of the sample in a single stage by adding to the sample a composition comprising the marking agent and one or several cellular penetration agents.

Such a process can be implemented, e.g., in accordance with the protocol described below:

Eight millimeters of the sample to be treated are incubated 60 minutes at ambient temperature with three millimeters of a cellular penetration/marking solution (PicoGreen 0.17 mL/l, PEI 20 mg/l, EDTA 4.34 g/l, nisine 0.47 mg/l, NOG 5.83 g/l, chlorohexidine diacetate 116/7 mg/l. The sample is then filtered on a char filter of polycarbonate and analyzed with a cytometer in solid phase.

The totality of these treatments can be realized indifferently in an open device, e.g., in tubes or in a closed device like a syringe or a device for the preparation of blood platelets for a bacteriological analysis (hemosystem, ref. SPK01).

Detection of Microorganisms

Determination of the Optimal Compositions of the Reaction Environment for the Marking/Cellular Penetration

1—Marking in the Presence of Nisine

The use of nisine solely as a permeabilizing agent for facilitating penetration of the marking agent.

I Reactants

Marking Solution

Prepare a solution of PicoGreen at 1/2000 (molecular probe) in PBS buffer (saline phosphate buffer, pH 7.4).

Nisine Solution

Prepare a series of dilutions of nisine (starting material at 2.5% weight/weight) in distilled water:
0.1 g nisine in 50 ml distilled water=solution 50 μg/ml,
0.02 g of nisine in 50 ml distilled water=solution 10 μg/ml,
0.004 g of nisine in 50 ml distilled water=solution 2 μg/ml.
   Suspension of Bacteria Prepared in PBS
Escherichia coli (CIP 105901)
Staphylococcus epidermidis (68.21)

Adjust the preparations in order to obtain a suspension with 10⁴ bacteria/ml.

II Method

1.2 ml of marking solution
+3 ml of bacterial suspension
Incubation 15 min at 22° C.
+7 ml of nisine solution
Filtration of char filter 0.4 μm porosity.
   III Analysis and Results

After the filtration, the filter is analyzed by cytometry in solid phase and the results expressed as the number of bacteria detected by cytometry in solid phase and in the percentage of bacteria detected relative to the method of enzymatic detection.

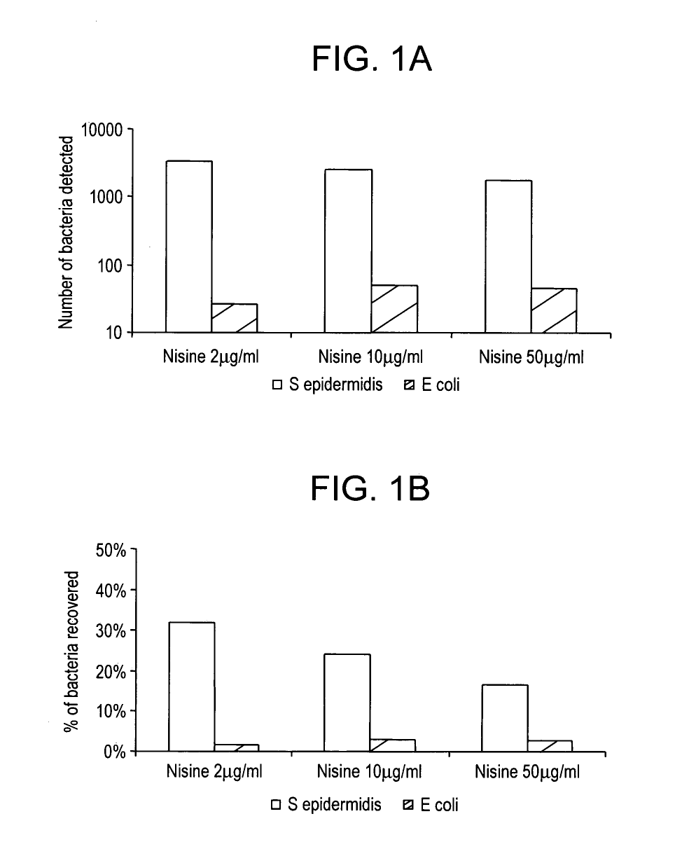

These results show the influence of the addition of nisine on the detection of Staphylococcus epidermidis and Escherichia coli and are illustrated in attached FIGS. 1A and 1B.

It can be determined that the addition of nisine permits the obtention of a good marking of the Gram+ and that low concentrations are preferable.
2—The Use of EDTA by Itself as a Permeabilizing Agent for Favoring Penetrating the Marking Agent
   I Reactants
   Marking Solution

Prepare a solution of PicoGreen at 1/2000 (molecular probe) in PBS buffer.

EDTA Solution

EDTA 5 mM: 0.093 g disodic EDTA, QSP 50 mL distilled water.

Suspension of bacteria prepared in PBS in distilled water in a TSB (tryptone soy broth) environment and in plasma.
Escherichia coli (CIP 105901)
Staphylococcus epidermidis (CIP 68.21)

Adjust the preparations to obtain a suspension with 10³ bacteria/ml.

II Method

1.2 mL of marking solution
+3 mL of bacterial suspension in the various environments
Incubation 15 minutes at 22° C.
+7 mL of EDTA solution
Filtration on char filter 0.4 μm porosity.
   III Analysis and Results

After filtration, the filter is analyzed by cytometry in solid phase and the results are expressed as the number of fluorescent bacteria.

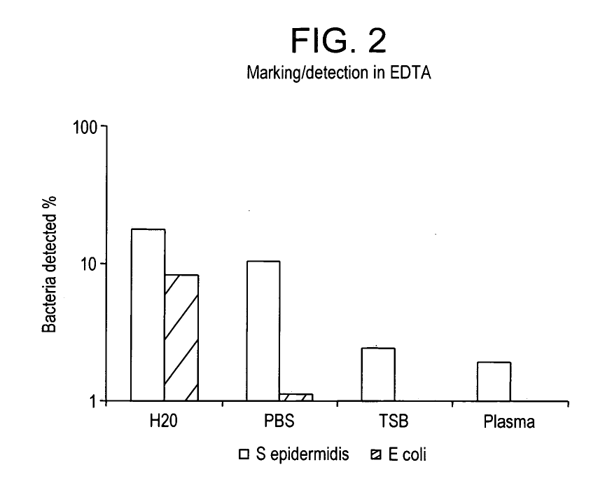

These results show the effect of EDTA used by itself for detecting Staphylococcus epidermidis and Escherichia coli prepared in different test environments and is illustrated in FIG. 2.

It can be determined that EDTA by itself does not permit a correct marking of Gram+ and Gram− bacteria.437
3—The Use of the Association Nisine/EDTA as Permeabilzing Agent for Favoring the Penetration of the Marking Agent
   I Reactants
   Marking Solution

Prepare a solution of PicoGreen at 1/2000 (molecular probe) in PBS buffer (saline phosphate buffer, pH 7.4).

Nisine/EDTA Solution

Nisine 10 μg/ml: 0.02 g nisine (starting material at 2.5% weight/weight) QSP 50 ml distilled water.
EDTA 20 mM: 0.372 g disodic EDTA, QSP 50 ml of distilled water,
Prepare a range of EDTA with 0.25; 2.5; 12.5 and 18.75 ml of EDTA 20 mM (concentration range 0.1; 1; 5 and 7.5 mM),
Add 50, 250, 500 μl or 1 ml nisine 10 μg/ml (concentration range 0.10; 0.05; 0.1 and 0.2 g/ml),
QSP 50 ml of distilled water.
   Suspension of Bacteria Prepared in PBS
Escherichia coli (CIP 105901)

Adjust the preparations to obtain a suspension with 10³ bacteria/ml.

II Method

1.2 ml of marking solution
+3 ml bacterial suspension
Incubation 15 minutes at 22° C.
+7 ml of solution of nisine or nisine/EDTA
Filtration on char filter 0.4 μm porosity.
   III Analysis and Results

After filtration, the filter is analyzed by cytometry in solid phase and the results expressed as the number of bacteria detected in cytometry in solid phase and as a percentage of bacteria detected relative to the method of enzymatic detection.

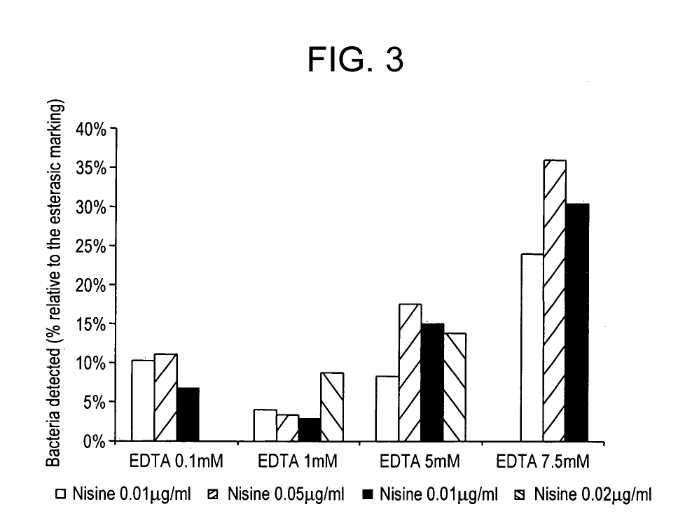

These results show the influence of the addition of nisine combined with EDTA on the detection of Escherichia coli and are illustrated in FIG. 3.

A synergistic effect on the detection of bacteria can be determined when the marking is carried out in the presence of the mixture nisine/EDTA. It can also be determined that the percentage of marked Escherichia coli bacteria is maximal for a concentration of nisine at 0.1 μg/ml and EDTA 7.5 mM.
4—Optimization of the Concentrations of the Association Nisine/EDTA as Cellular Penetration Reactant for Detecting Bacteria
   I Reactants
   Marking Solution

Prepare a solution of PicoGreen at 1/2000 (molecular probe) in PBS buffer (saline phosphate buffer, pH 7.4).

Nisine/EDTA Solution

0.2 g nisine (starting material at 2.5% weight/weight) in 50 ml distilled water or 10 μg/ml,

Nisine 0.05 μg/ml/EDTA 7.5 mM: 250 μl nisine 10 μg/ml +0.140 g disodic EDTA, QSP 50 mL distilled water,

Nisine 0.1 μg/ml/EDTA 7.5 mM: 500 μa l nisine 10 μg/ml +0.140 g disodic EDTA, QSP 50 mL distilled water,

Nisine 0.5 μg/ml/EDTA 7.5 mM: 2.5 ml nisine 10 μg/ml+0.140 g disodic EDTA, QSP 50 mL water.

Suspension of Bacteria Prepared in PBS

Escherichia coli (CIP 105901)
Staphylococcus epidermidis (CIP 68.21)
Serratia marcescens (CIP 103716)

Adjust the preparations to obtain a suspension with 10³ bacteria/ml.

II Method

1.2 mL marking solution
+3 mL bacterial suspension
Incubation 15 minutes at 22° C.
Incubation 15 minutes at 22° C.
+7 ml solution of nisine/EDTA at different concentrations
Filtration on char filter 0.4 μm porosity.
   III Analysis and Results

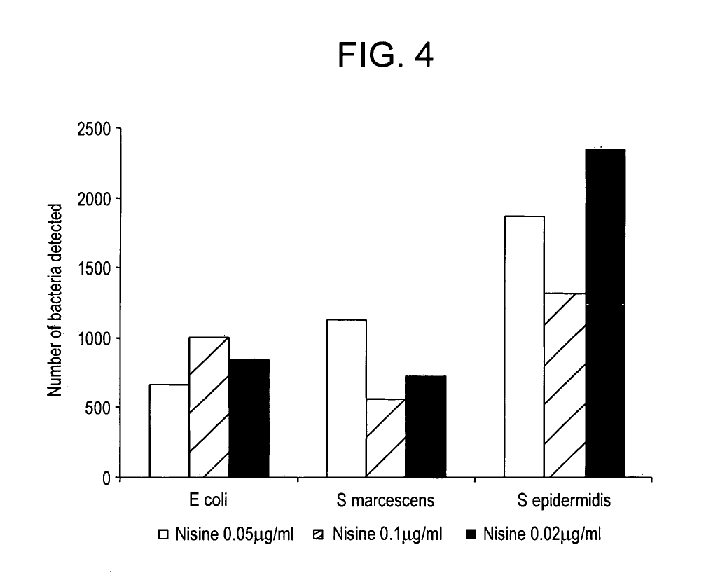

After filtration, the filter is analyzed by cytometry in solid phase and the results expressed as the number of bacteria detected in cytometry in solid phase. The results of this experiment showing the detection of the bacteria Gram (−) (Escherichia coli, Serratia marcescens) and Gram+ (Staphylococcus epidermidis) in the presence of different concentrations of nisine associated with EDTA 7.5 mM are illustrated in FIG. 4.

It can be confirmed that the percentage of marked Escherichia coli bacteria is maximal for a concentration of nisine at 0.1 μg/ml and that a better detection of the entirely of bacteria tested is obtained when nisine is used at a concentration of 0.2 μg/ml associated with EDTA at a concentration of 7.5 mM.
5—Influence of the pH on the Marking of Bacteria in the Presence of Nisine/EDTA
   I Reactants
   Marking Solution

Prepare a solution of PicoGreen at 1/2000 (molecular probe) in PBS buffer (saline phosphate buffer, pH 7.4).

Nisine/EDTA Solution

Nisine 10 μg/ml: 0.02 g nisine (starting material at 2.5% weight/weight) QSP 50 ml distilled water,
EDTA 20 mM: 0.372 g disodic EDTA, QSP 50 ml distilled water,
18.75 ml EDTA 20 mM,
+1 ml nisine 10 μg/ml.
This solution is at pH 4.8
Buffer with NaOH (1 M) until pH 6, pH 7, pH 8.
Suspension of bacteria prepared in PBS
Escherichia coli (CIP 105901)
Staphylococcus epidermidis (CIP 68.21)
Serratia marcescens (CIP 103716)
Enterobacter aerogenes (CIP 60.86T)
Pseudomonas aeruginosa (CIP 76110)
Proteus mirabilis (CIP 104588)

Adjust the preparations to obtain a suspension with 10³ bacteria/ml.

II Method

1.2 mL marking solution
+3 mL bacterial suspension
Incubation 15 minutes at 22° C.
+7 mL solution of nisine/EDTA at different pH'es
Filtration on char filter 0.4 μm porosity.
   III Analysis and Results

After filtration, the filter is analyzed by cytometry in solid phase and the results expressed as the number of fluorescent bacteria detected.

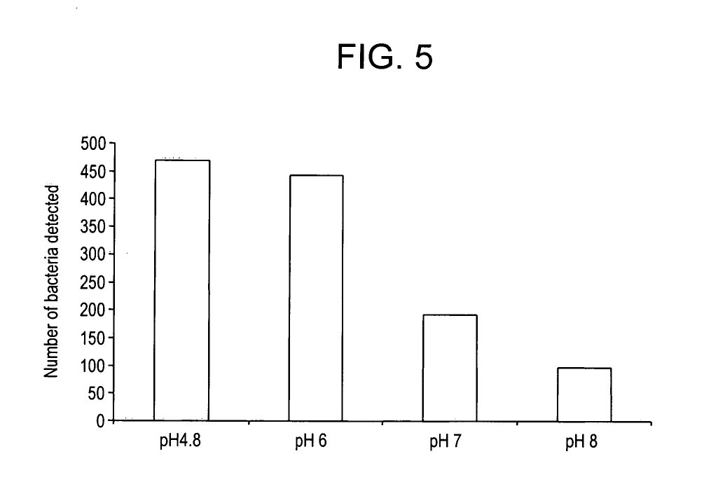

The results of this experiment showing the influence of the pH on the detection of Escherichia coli with a fluorescent marker of DNA in the presence of nisine 0.2 μg/ml/EDTA 7.5 mM are illustrated in FIG. 5.

It can be confirmed that under the predefined conditions the increasing of the pH does not improve the marking of Escherichia coli.

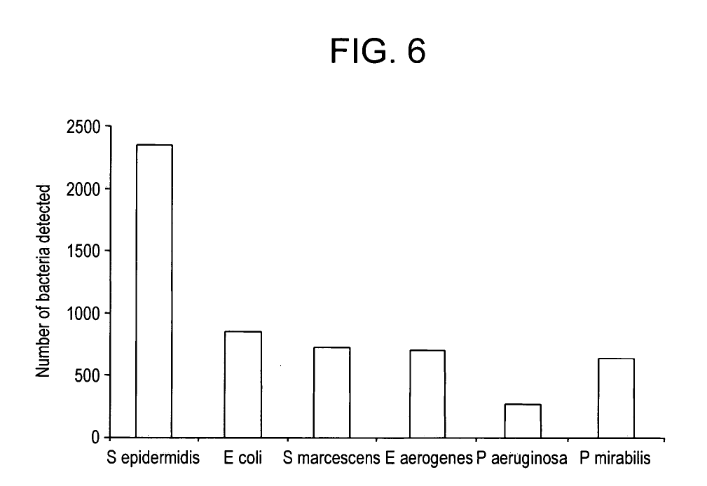

The detection of the Gram+ bacteria Staphylococcus epidermidis and Gram− Escherichia coli, Serratia marcescens, Enterobacter aerogenes, Pseudomonas aeruginosa, Proteus mirabilis with a fluorescent marker of DNA in the presence of nisine 0.2 μg/ml/EDTA 7.5 mM at pH 4.8 is illustrated in FIG. 6.

It can be confirmed that under the conditions of pH at 4.8 the marking of Gram (−) bacteria is homogeneous from one strain to the other. The detection of Gram (+) Staphylococcus epidermidis is more elevated than that of the Gram (−).
6—Association Nisine/EDTA/N Octyl Glucopyranoside
   I Reactants
   Marking Solution

Prepare a solution of PicoGreen at 1/2000 (molecular probe) in PBS buffer (saline phosphate buffer, pH 7.4).
Nisine/EDTA/NOG solution
Nisine 100 μg/ml: 0.02 g nisine (starting material at 2.5% weight/weight) QSP 50 ml distilled water.
EDTA 100 mM: 1/86 g disodic EDTA, QSP 50 ml distilled water,
N octyl glucopyranoside 5%: 2.5 g in 50 ml distilled water
20, 10, 5 or 2.5 ml NOG at 5%
+3.75 ml EDTA 100 mM
+0.1 ml nisine 100 μg/ml
QSP 50 ml distilled water
This solution is at pH 4.8.
   Suspension of Bacteria Prepared in PBS
Staphylococcus epidermidis (CIP 68.21)
Pseudomonas aeruginosa (CIP 76110)

Adjust the preparations to obtain a suspension with 10³ bacteria/ml.

II Method

1.2 ml of marking solution
+3 ml of bacterial suspension
Incubation 15 minutes at 22° C.
+7 ml of solution of nisine/EDTA or nisine/EDTA/NOG
Filtration on char filter 0.4 μm porosity.
   III Analysis and Results

After filtration, the filter is analyzed by cytometry in solid phase and the results expressed as the number of fluorescent bacteria.

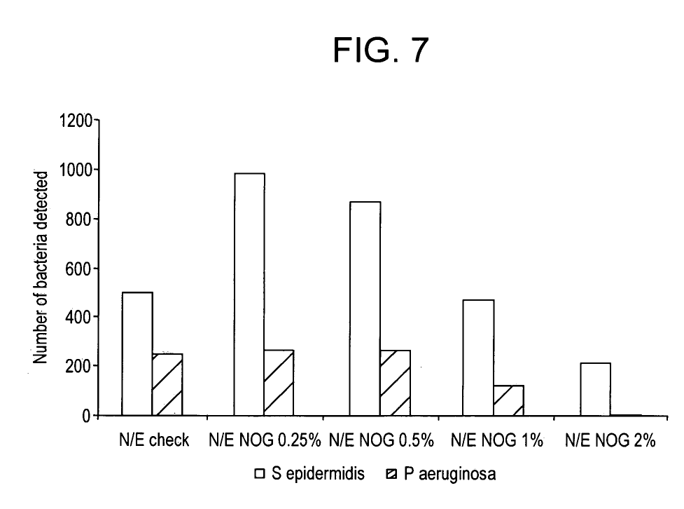

The results of this experiment with a composition of the reaction environment associating N octyl glucopyranoside as cellular penetration reactant of the microorganisms with nisine 0.2 μg/ml and EDTA 7.5 mM for improving the marking of Staphylococcus epidermidis (Gram+) and Pseudomonas aeruginosa (Gram−) are illustrated in FIG. 7.

It can be confirmed that the addition of N octyl glucopyranoside at 0.25% and at 0.5% has positive effects on the marking of Staphylococcus epidermidis and of Pseudomonas aeruginosa.
7—Marking in the Presence of Chlorohexidine

Test implementing chlorohexidine only as permeabilizing agent for favoring the penetration of the bacterial marking agent.

I Reactants

Marking Solution

Prepare a solution of PicoGreen at 1/2000 (molecular probe) in PBS buffer (saline phosphate buffer, pH 7.4).

Solution of Chlorohexidine

Diacetate chlorohexidine 5%: 1 g in 20 mL distilled water 50, 25 or 10 μl diacetate chlorohexidine 5% in 50 mL distilled water in order to obtain a concentration range of 0.01%; 0.005% or 0.001%.
   Suspension of Bacteria Prepared in PBS, in Platelet Concentrate and Autologous Plasma
Escherichia coli (CIP 105901)
Staphylococcus epidermidis (CIP 68.21)
Serratia marcescens (CIP 103716)
Pseudomonas aeruginosa (CIP 76110)

Adjust the preparations to obtain a suspension with 10³ bacteria/ml.

II Method

1.2 mL marking solution
+3 mL bacterial suspension
Incubation 15 minutes at 22° C.
+7 mL of chlorohexidine solution at different concentrations
Filtration on char filter 0.4 μm porosity.
   III Analysis and Results

After filtration, the filter is analyzed by cytometry in solid phase and the results expressed as the number of fluorescent bacteria. The counting on a Petri dish at 48 hours takes place with the reference method.

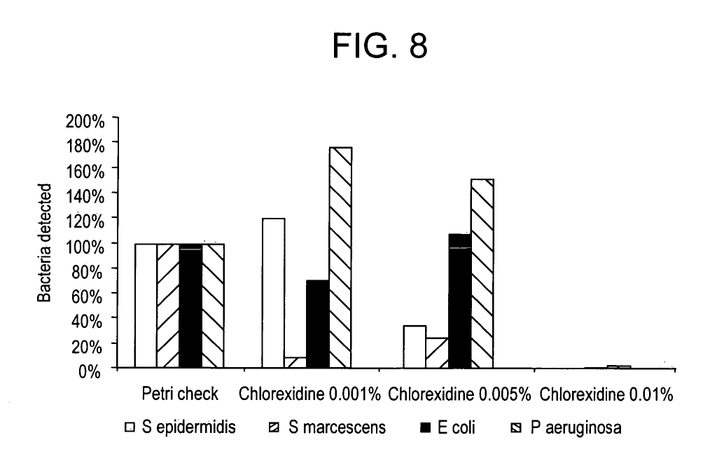

The results of this experiment with a composition of the reaction environment comprising chlorohexidine as cellular penetration reactant to improve the marking of Escherichia coli, Pseudomonas aeruginosa and Serratia marcescens (Gram (−) bacterial strains) and Staphylococcus epidermidis are illustrated in FIG. 8.

It can be confirmed that the optimal concentration of chlorohexidine for the detection of Gram− bacteria is 0.005%. However, this concentration is toxic for Gram+ bacteria, that are destroyed.

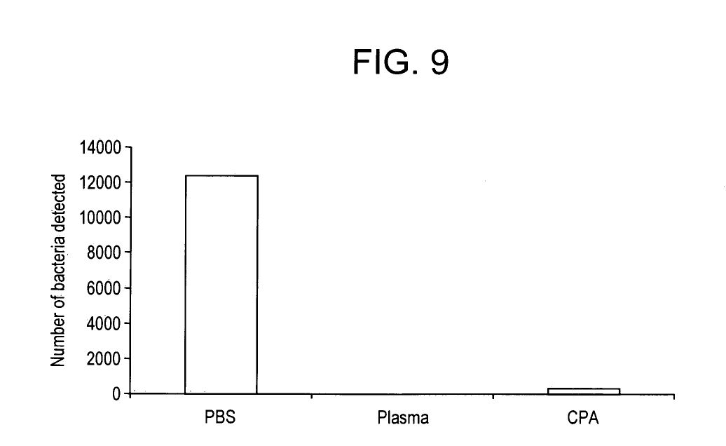

It is confirmed that the presence of plasma antagonizes the effect of chlorohexidine on the cellular penetration of the marker for Pseudomonas aeruginosa as illustrated in FIG. 9. For a universal marking in different environments including the biological fluids, chlorohexidine alone can not be used.
8—Association chlorohexidine/N-octyl glucopyranoside
   I Reactants
   Marking Solution

Prepare a solution of PicoGreen at 1/2000 (molecular probe) in PBS buffer (saline phosphate buffer, pH 7.4).

Solution of Chlorohexidine/N Octyl Glucopyranoside

Diacetate chlorohexidine 5%: 1 g in 20 ml distilled water
N octyl glucopyranoside 1%: 0.5 g in 50 ml distilled water
50 or 25 μl diacetate chlorohexidine 1% (final concentration of 0.001% or 0.0005%)
QSP 50 ml distilled water.
   Suspension of Bacteria Prepared in PBS and in Platelet Concentrate of Apheresis
Escherichia coli (CIP 105901)
Staphylococcus epidermidis (CIP 68.21)
Serratia marcescens (CIP 103716)
Pseudomonas aeruginosa (CIP 76110)

Adjust the preparations to obtain a suspension with 10³ bacteria/ml.

II Method

1.2 mL marking solution
+3 mL bacterial suspension
Incubation 15 minutes at 22° C.
+7 mL of chlorohexidine/NOG solution
Filtration on char filter 0.4 μm porosity.
   III Analysis and Results

After filtration the filter is analyzed by cytometry in solid phase and the results expressed as the number of fluorescent bacteria.

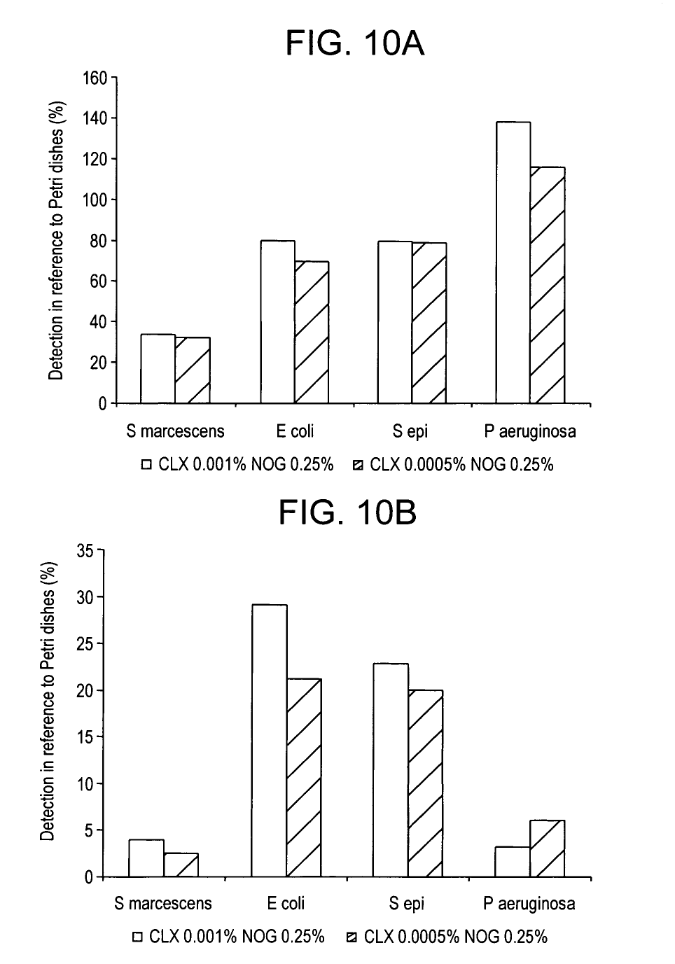

The results of this experiment showing the marking of DA and the detection of marked bacteria in the presence of a composition of the reaction environment comprising chlorohexidine in association with NOG for increasing the permeabilizing power and the penetration of the marker are illustrated in FIGS. 10A and 10B.

The most elevated concentration of chlorohexidine permits the obtention of the best marking of the bacteria.
9—Marking in the Presence of Only PEI
   I Reactants
   Marking Solution

Prepare a solution of PicoGreen at 1/2000 (molecular probe) in PBS buffer (saline phosphate buffer, pH 7.4) and add PEI for a final concentration of 40, 80, 100, 120, 140 and 160 μg/ml.

Bacterial Suspension Prepared in PBS

Serratia marcescens (CIP 103716)
Sample analyzed
Dilution>1/20 of the bacterial suspension in a sample of platelet concentrate for obtaining a final bacterial concentration of Serratia marcescens of 10⁴/ml.
   II Method
1.2 mL of marking solution
+3 mL sample incubation 45 minutes at 23° C.
Filtration 5 μm (PALL filters 32 mm)
Incubation 20 minutes in 7 mm PBS
Filtration 0.4 μm porosity.
   III Analysis and Results

After filtration, the filter is analyzed by cytometry in solid phase and the results expressed as the number of fluorescent bacteria detected.

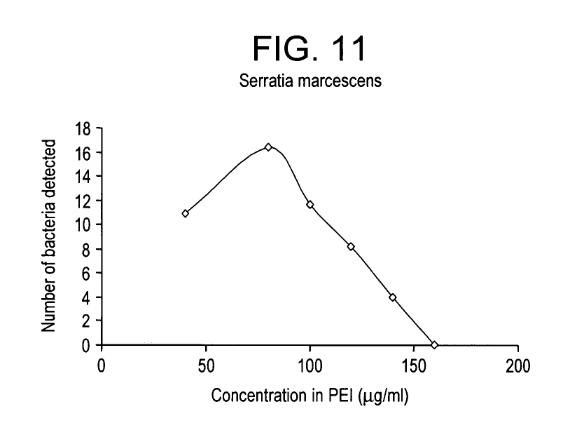

The results of this experiment showing the effect of different concentrations of PEI on the detection of Serratia marcescens with a fluorescent DNA marker are illustrated in FIG. 11.

It can be confirmed that an optimal detection of bacteria is obtained with a concentration range of PEI comprise between 40 and 100 μg/ml.
10—Association Nisine/EDTA/N Octyl Glucopyranoside/Chlorohexidine/PEI

The objective of this experiment is to determine the optimal concentration range in PEI for the marking of Escherichia coli.

I Reactants

Marking Solution

Prepare a solution of PicoGreen at 1/2000 (molecular probe) in PBS buffer (saline phosphate buffer, pH 7.4) and add PEI for a final concentration of 100, 80 and 60 μg/ml. Solution of chlorohexidine/N octyl glucopyranoside/EDTA
500 ml of diacetate chlorohexidine at 0.5% (diacetate chlorohexidine 5×10³% final)
+1 ml or 500 μL N octyl glucopyranoside 25% (N octyl glucopyranoside 0.5 or 0.25% final)
+500 μL nisine 20 μg/ml (nisine 0.2 μg/ml final)
QSP 50 ml PBS.
   Suspension of Bacteria Prepared in PBS
Escherichia coli (CIP 105901), adjustment of the concentration to 10⁴ bacteria/ml. Analyzed sample

3 ml of bacterial suspension +27 ml of platelet concentrate or a dilution at 1/10 of the bacterial suspension in a sample of platelet concentrate for obtaining a final bacterial concentration of 10⁵/ml.

II Method

1.2 mL of marking solution at 60, 80 or 100 μg/ml PEI
+3 mL sample
Incubation 45 minutes at 23° C.
Filtration 5 μm (PALL filters 32 mm)
Incubation 20 minutes in 7 mm cellular penetration solution at 0.5% or 0.25% NOG
Filtration 0.4 μm (Whatman monocolor char filters).
   III Analysis and Results

After filtration, the filter is analyzed by cytometry in solid phase and the results expressed as the number of fluorescent bacteria detected.

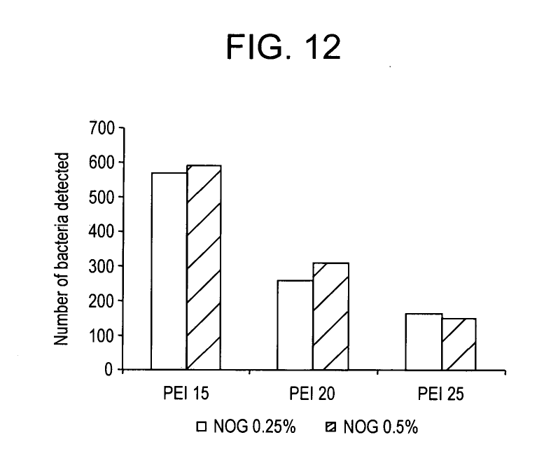

The results of this experiment showing the effect of PEI on DNA marking and the detection of Escherichia coli in fluorescence are illustrated in FIG. 12.

It can be confirmed that the concentration of 60 μg/ml of PEI permits an optimal penetration of the DNA marker whatever the concentration of NOG.
11—Universal Marking of Bacteria in Different Environments
   I Reactants
   Marking Solution

Prepare a solution of PicoGreen at 1/2000 (molecular probe) in PBS buffer (saline phosphate buffer, pH 7.4) and add PEI for a final concentration of 60 μg/ml. Solution of chlorohexidine/N octyl glucopyranoside/EDTA/nisine
500 ml of diacetate chlorohexidine at 0.5% (diacetate chlorohexidine 5×10⁻³% final)
+500 μL N octyl glucopyranoside 25% (N octyl glucopyranoside 0.25% final)
+500 μL nisine 20 μg/ml (nisine 0.2 μg/ml final)
+500 μl EDTA 0.5 M (EDTA 5 mM final)
QSP 50 ml PBS.
   Suspension of Bacteria Prepared in PBS
Escherichia coli (CIP 105901), adjustment of the concentration to 10⁴ bacteria/ml.
Staphylococcus epidermidis (68.21).
   Analyzed Sample

Dilution at 1/10 of the bacterial suspension in a sample of biological fluid for obtaining a final bacterial concentration of 10³/ml or:
3 ml of bacterial suspension+27 mL distilled water
3 ml of bacterial suspension+27 mL PBS
3 ml of bacterial suspension+27 mL culture environment (tryptone soy broth)
3 ml of bacterial suspension+27 mL human plasma
3 ml of bacterial suspension+27 mL platelet concentrate.
   II Method
1.2 mm of marking solution +3 mL sample
Incubation 45 minutes at 23° C.
Filtration 5 μm, incubation 20 minutes in 7 mL of cellular penetration solution.
Filtration 0.4 μm porosity.
   III Analysis and Results

After filtration, the filter is analyzed by cytometry in solid phase and the results expressed as the number of fluorescent bacteria detected.

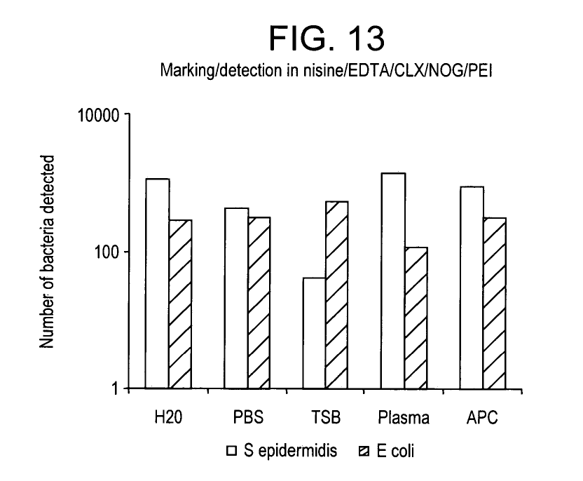

The results of this experiment showing the detection of Staphylococcus epidermidis and Escherichia coli in different environments are illustrated in FIG. 13.

The formula defined in this manner permits the detection of Gram+ and Gram (−) bacteria in different ionic, culture and physiological environments. This detection is comparable for the two types of bacteria.

### BIBLIOGRAPHIC REFERENCES

Saitoh H, Yamane N. Comparative evaluation of BACTEC MGIT 960 system with MB/BacT and egg-based media for recovery of mycobacteria. Rinsho Biseibutshu Jinsoku Shindan Kenkyukai Shi 2000 August; 11(1): 19-26.

Brecher M E, Means N, Jere C S, Heath D, Rothenberg S, Stutzman L C. Evaluation of an automated culture system for detecting bacterial contamination of platelets: an analysis with 15 contaminating organisms. Transfusion. 2001 April; 41(4):477-82.

Brecher M E, Heath D G, Hay S N, Rothenberg S J, Stutzman L C. Evaluation of a new generation of culture bottle using an automated bacterial culture system for detecting nine common contaminating organisms found in platelet components. Transfusion 2002 June; 42(6):774-9.

He Q, Wang J P, Osato M, Lachman L B. Real-Time Quantitative PCR for Detection of Helicobacter pylori. J Clin Microbiol 2002 October; 40(10):3720-8.

Al-Soud W A, Jonsson L J, Radstrom P. Identification and characterization of immunoglobulin G in blood as a major inhibitor of diagnostic PCR. J Clin Microbiol 2000 January; 38(1):345-50.

Guamer J, Shieh W J, Greer P W, Gabastou J M, Chu M, Hayes E, Nolte K B, Zaki S R. Immunohistochemical detection of Yersinia pestis in formalin-fixed, paraffin-embedded tissue. Am J Clin Pathol 2002 Febuary; 117(2):205-9.

Kakinoki K, Takemori Y, Noda Y. Efficacy of the urine antibody test for detection of Helicobacter pylori: comparison with serum antibody tests. Nippon Shokakibyo Gakkai Zasshi 2001 August; 98(8):935-41.

Poppert S, Essig A, Marre R, Wagner M, Horn M. Detection and differentiation of chlamydiae by fluorescence in situ hybridization. Appl Environ Microbiol 2002 August; 68(8):4081-9.

Braun-Howland E B, Danielsen S A, Nierzwicki-Bauer S A. Development of a rapid method for detecting bacterial cells in situ using 16S rRNA-targeted probes. Biotechniques 1992 December; 13(6):928-34.

Fazii P, Ciancaglini E, Riario Sforza G. Differential fluorescent staining method for detection of bacteria in blood cultures, cerebrospinal fluid and other clinical specimens. Eur J Clin Microbiol Infect Dis 2002 May; 21(5):373-8.

Mirrett S, Lauer B A, Miller G A, Reller L B. Comparison of acridine orange, methylene blue, and Gram stains for blood cultures. J Clin Microbiol 1982 April; 15(4):562-6.

Reynolds D T, Fricker C R. Application of laser scanning for the rapid and automated detection of bacteria in water samples. J Appl Microbiol 1999 May; 86(5):785-95.

Aubert G, Vautrin A C, Michel V P, Fresard A, Dorche G. Evaluation of three automated blood culture systems. Bio Argod, Bact T/Alert, bactec NR-860. Pathol Biol (Paris) 1993 April; 41(4):434-40.

Okada H, Sakai Y, Miyazaki S, Arakawa S, Hamaguchi Y, Kamidono S. Detection of significant bacteriuria by automated urinalysis using flow cytometry. J Clin Microbiol 2000 August; 38(8):2870-2.

Marie D, Vaulot D, Partensky F. Application of the novel nucleic acid dyes YOYO-1, YO-PRO-1, and PicoGreen for flow cytometric analysis of marine prokaryotes. Appl Environ Microbiol 1996 May; 62(5):1649-55.

Wallner G, Tillmann D, Haberer K, Comet P, Drocourt J-L. The Chemscan system: a new method for rapid microbiological testing of water. European Journal of Parenteral Sciences 1997; 2(4):123-26.

1. A process for detecting microorganisms present in a biological fluid comprising:
a) contacting a sample of the biological fluid with a reaction environment comprising a marking agent that is a derivative of cyanines and at least one reactant of cellular penetration of the membrane of the microorganisms,
b) filtering the sample on a filter capable of retaining the marked microorganisms present in the sample, and
c) detecting the marked microorganisms retained in the filter in stage (b).

2. The process according to claim 1, wherein the cyanine derivatives are selected from the group consisting of PicoGreen, SYBR green and YOPRO1 and are present in an amount between about 0.001% and about 0.5%.

3. The process according to claim 1, wherein the cellular penetration agent of the microorganisms is selected from the group consisting of a detergent, an enzyme, a bacteriocine, an ion chelating agent, a fixation agent, a permeabilization agent and mixtures thereof.

4. The process according to claim 3, wherein the detergent is selected from the group consisting of N-octyl β D-glucopyranoside (NOG), saponine, Tween, Triton, Igepal, CHAPS and mixtures thereof.

5. The process according to claim 4, wherein the concentration of saponine or of Tween is between about 0.005% and about 10%, the concentration of NOG is between about 0.01% and about 10%, the concentration of Triton is between about 0.0001% and about 0.05%, and the concentration of Igepal is between about 0.01% and about 20%.

6. The process according to claim 3, wherein the enzyme is lysozyme in a concentration between about 0.5 μg/ml and about 200 μg/ml.

7. The process according to claim 3, wherein the bacteriocine is nisine in a concentration between about 0.005 μg/ml and about 200 μg/ml.

8. The process according to claim 3, wherein the ion chelating agent is selected from the group consisting of EDTA and EGTA.

9. The process according to claim 8, wherein the concentration of EDTA is between about 0.5 mM and about 50 mM.

10. The process according to claim 3, wherein the fixation agent is selected from the group consisting of formaldehyde, paraformaldehyde, glutaraldehyde, ethanol, streptolysine O, osmium tetroxide, orthophthalaldehyde and mixtures thereof.

11. The process according to claim 10, wherein the concentration of formaldehyde or glutaraldehyde is between about 0.05% and about 10%, the concentration of ethanol or streptolysine O is between about 0.1% and about 20%, and the concentration of osmium tetroxide or orthophthalaldehyde is between about 0.005% and about 10%.

12. The process according to claim 3, wherein the permeabilizing agent is selected from the group consisting of polyethylene glycol (PEG), digitonine, monensine, polyethylenimine (PEI), sodium hexamethaphosphate, benzalkonium chloride and mixtures thereof.

13. The process according to claim 12, wherein the concentration of PEG is between about 0.01% and about 1%, the concentration of digitonine is between about 0.01 μg/ml and about 10 μg/ml, the concentration of monensine is between about 0.1 μg/ml and about 5 μg/ml, the concentration of PEI is between about 1 μg/ml and about 400 μg/ml, the concentration of sodium hexametaphosphate is between about 0.005% and about 1%, and the concentration of benzalkonium chloride is between about 0.001% and about 0.1%.

14. The process according to claim 1, wherein the composition of the reaction environment further comprises:
an antibiotic agent selected from the group consisting of polymixine B, rifampicine and mixtures thereof;
an antiseptic agent selected from the group consisting of Betadine, cetrimide, tea plant oil, terpinene-4-ol, chlorohexidine and mixtures thereof; and
a mixture of the antibiotic agent and the antiseptic agent.

15. A reaction environment for marking microorganisms, comprising a marking agent that is a derivative of cyanines and at least one cellular penetration agent of the microorganisms.

16. The reaction environment according to claim 15, wherein that the cellular penetration agent is selected from the group consisting of a detergent, an enzyme, a bacteriocine, an ion chelating agent, a fixation agent, a permeabilization agent and mixtures thereof.

17. The reaction environment according to claim 16, wherein the detergent is selected from the group consisting of N-octyl β D-glucopyranoside (NOG), saponine, Tween, Triton, Igepal, CHAPS and mixtures thereof.

18. The reaction environment according to claim 16, wherein the enzyme is lysozyme.

19. The reaction environment according to claim 16, wherein the bacteriocine is nisine.

20. The reaction environment according to claim 16, wherein the ion chelating agent is selected from the group consisting of EDTA, EGTA and mixtures thereof.

21. The reaction environment according to claim 16, wherein the fixation agent is selected from the group consisting of formaldehyde, paraformaldehyde, glutaraldehyde, ethanol, streptolysine O, osmium tetroxide, orthophthalaldehyde and mixtures thereof.

22. The reaction environment according to claim 16, wherein the permeabilizing agent is selected from the group consisting of polyethylene glycol (PEG), digitonine, monensine, polyethylenimine (PEI), sodium hexamethaphosphate, benzalkonium chloride and mixtures thereof.

23. The reaction environment according to claim 15, further comprising:
an antibiotic agent selected from the group consisting of polymyxine B, rifampicine and mixtures thereof,
an antiseptic agent selected from the group consisting of Betadine, cetrimide, tea plant oil, terpinene-4-ol, chlorohexidine and mixtures thereof, and
a mixture of the antibiotic agent and the antiseptic agent.

24. A process for detecting microorganisms present in a biological fluid comprising:
a) contacting a sample of the biological fluid with a reaction environment for marking of the microorganisms comprising a marking agent and a reactant of cellular penetration of the membrane of the microorganisms,
b) filtering the sample on a filter capable of retaining the marked microorganisms present in the sample, and
c) detecting the marked microorganisms retained in the filter in stage (b).

25. The process according to claim 24, wherein the marking agent is an intercalator compound of DNA.

26. The process according to claim 25, wherein the intercalator DNA agent is selected from the group consisting of Cyanine compounds, propidium iodide, orange acridine, ethidium bromide and mixtures thereof.

27. The process according to claim 24, wherein stage a) comprises two sub-stages a′) and a″), of which stage a′) comprises placing the sample in contact with a reaction environment comprising a marking agent and a permeabilizing polymer selected from the group consisting of polyethylene glycol (PEG) and polyethylenimine (PEI) and stage a″) comprises adding a mixture to the reaction environment, which mixture comprises at least one of a detergent, an ion chelating agent, an antiseptic and another permeabilizing agent selected from the group consisting of nisine, digitonine, sodium hexamethaphosphate, benzalkonium chloride and mixtures thereof.

28. A cellular penetration reactant comprising:
PicoGreen at 1/22000 (molecular probes);
PEI at a final concentration of about 5.5 μg/ml;
Diacetate chlorohexidine at a final concentration of about 4.5×10⁻⁴%;
N octyl glucopyranoside at a final concentration of about 0.16%;
Nisine at a final concentration of about 0.018 μg/ml;
EDTA at a final concentration of about 0.45 mM; and
a buffer saline phosphate (PPS) in a quantity sufficient for a selected final volume.
